(19) United States
(12) Patent Application Publication
(10) Pub. No.: US 2024/0342489 A1
(43) Pub. Date: Oct. 17, 2024

(54) METHOD FOR IEGM-BASED MONITORING OF AN ELECTRODE STATUS OF AN IMPLANTABLE DEVICE

(71) Applicant: BIO TRONIK SE & Co. KG, Berlin (DE)

(72) Inventors: René FISCHER, Berlin (DE); Peter LIEBISCH, Berlin (DE); Jens KNUEPPEL, Berlin (DE); Katrin WOLFF, Berlin (DE)

(73) Assignee: BIO TRONIK SE & Co. KG, Berlin (DE)

(21) Appl. No.: 18/682,186

(22) PCT Filed: Jul. 12, 2022
(86) PCT No.: PCT/EP2022/069464

§ 371 Date: Feb. 8, 2024

(30) Foreign Application Priority Data
Aug. 9, 2021 (EP) 21190241.6

(51) Int. Cl. A61N 1/37; A61N 1/02

(52) U.S. Cl. CPC A61N 1/3704; A61N 1/025; A61N 1/3706

(57) ABSTRACT

A method for monitoring an implantable device. The method comprises: receiving at least one cardiac vector signal, wherein the at least one cardiac vector signal is acquired by the implantable device between at least one pair of electrodes; extracting two or more separate features from the at least one cardiac vector signal by signal processing; deriving a hardware status (pₙₒᵢₛₑ) of the implantable device based at least in part on a classification of the extracted two or more separate features.

### CROSS-REFERENCE TO RELATED APPLICATIONS

This application is the United States National Phase under 35 U.S.C. § 371 of PCT International Patent Application No. PCT/EP2022/069464, filed on Jul. 12, 2022, which claims the benefit of European Patent Application No. 21190241.6, filed on Aug. 9, 2021, the disclosures of which are hereby incorporated by reference herein in their entireties.

### TECHNICAL FIELD

The present invention generally relates to a method for monitoring an implantable device (e.g., a cardiac implant), a corresponding implantable device, a corresponding system and a corresponding computer program.

### BACKGROUND

Various methods for monitoring an implantable device (e.g., an implantable cardiac implant, e.g., for regulating a heart activity) are currently known which may be used to track its functionality when implanted into a patient. Usually a malfunction of the implantable device needs to be detected as early as possible before the malfunction may cause harm to the patient (e.g., a defective cardiac implant may falsely trigger or inhibit electrical stimulations to the heart causing medical harm). Therefore, a hardware status (e.g., the technical properties) of the implantable device may be regularly determined to detect a faulty hardware status before a medically critical hardware status (i.e., a status harmful to the patient) is imminent. The hardware status of a cardiac implant, for example, may comprise the state of the electrodes, the state of the leads, the fixation properties to the body tissue, external interferences, loose screws for the connection of the implantable device case with the leads, etc. of the implantable device.

A common technique for monitoring the hardware status may be a regular occurring impedance measurement between a pair of electrodes of the implantable device. This method may require the output of (electrical) pulses via the electrodes such that an impedance measurement may be performed. This approach suffers from straining the (electrical) power resources of the implant since it requires (electrical) energy to determine the hardware status. Thus, this approach limits device longevity. Additionally, such impedance measurements are not performed very frequently in order to minimize the stated effects on the device's longevity. Further, the reaction time between the detection of a faulty hardware status and the medically critical hardware status may be rather short in this approach.

Another monitoring technique may be analyzing timing-based criteria of a determined heart activity. The heart activity may be detected by common algorithms wherein the associated time span of the heart activity is analyzed for determining the hardware status. For example, if a physiological unusual time signature of a particular heart activity (e.g., a ventricular tachycardia) is detected, it may correlate with a (faulty) hardware status.

Further monitoring techniques may be based on the analysis of a sensing amplitude or an analysis of the pacing threshold of the implantable device.

The currently known techniques, however, do not always lead to an optimal monitoring of the implantable device. Therefore, there is a need to find ways to improve the monitoring of an implantable device.

The aspects describe herein address the above need at least in part. The present disclosure is directed toward overcoming one or more of the above-mentioned problems, though not necessarily limited to embodiments that do.

### SUMMARY

A first aspect relates to a method for monitoring an implantable device which may comprise receiving at least one cardiac vector signal, wherein the at least one cardiac vector signal is acquired by the implantable device between at least one pair of electrodes. The method may further comprise extracting two or more separate features from the at least one cardiac vector signal by signal processing. The method may further comprise deriving a hardware status of the implantable device based at least in part on a classification of the extracted two or more separate features.

The present invention may be based on the concept that the hardware status, a hardware deviation and/or disturbance caused by other (non-physiological) origins of an implantable device may correlate with the characteristics of the at least one cardiac vector signal. For example, the hardware status may influence the acquired at least one cardiac vector signal (e.g., the hardware status may be related to a bad interface contact which leads to a disturbed signal pickup of the at least one cardiac vector signal). A deviation of a hardware status from the norm may thus reciprocate as a “hidden” systemic input to the at least one cardiac vector signal. In this regard, the extraction of two or more separate features of the at least one cardiac vector signal may effectively enable the derivation of the hardware status. Hence, the underlying idea of the inventive concept may be based on classifying the extracted two or more separate features into a classification group. A particular classification group may be defined by certain classification criteria that the extracted two or more features have to meet to be associated with said classification group. In accordance with the present invention, a classification group (and its corresponding classification criteria) may be associated with a hardware status of the implantable device. Hence, the classification result (e.g., the classification group that the extracted two or more separate features fall into) may be used to derive a hardware status, a hardware deviation from the norm of the implantable device, and/or disturbance caused by other (non-physiological) origins. Since at least two separate features are extracted from the vector signals and used for analysis, the hardware status may be derived reliably, even in cases in which a single feature would be ambiguous.

The present invention may further resolve the drawbacks of common monitoring methods since it may not suffer from their known disadvantages, as e.g., high energy consumption, low sensitivity. In some examples, no impedance measurement may be needed. Usually, the known approaches may be too reactive because the hardware deviation may be detected not until there are already physical consequences for the patient (e.g., false administering of electrical shocks to the patient by the implant). Notably, the inventive concept may reduce energy consumption since it does not require outputting a specific electrical pulse for an impedance measurement which may drain an energy supply. Further, it may enable an improved detection performance (e.g., increased detection sensitivity) since the inventive method may be inherently constructed and/or optimized for deriving a hardware status. Notably, a common monitoring technique may suffer from a limited detection performance since it modifies an algorithm for detecting heart activities to detect timing-based artefacts of the heart activity (e.g., a cluster of short, physiologically unexplainable heart activity intervals). These timing-based artefacts may be associated and/or correlated with a hardware deviation. This known approach may suffer from the requirement that the sensing unit of the implantable device must detect the sensing artefacts while simultaneously sensing high amplitude physiological signals thus leading to a limited sensitivity.

In contrast, the inventive method may enable arbitrarily tuning the method regarding energy consumption, high sensitivity, early detection of a hardware status, range of derived hardware statuses and/or hardware deviations, etc. For example, the method may be arbitrarily scaled for a specific application by setting a number of extracted features for generating a specific feature vector, the types of extracted features, a number of classification groups, a type of classification group, the characteristics of the classification criteria, the amount of cardiac vector signals, types of signal processing applied to the at least one cardiac vector signal, etc. Hence, the inventive method may enable an improved approach for detecting a hardware malfunction in implantable devices due to the available wide degree of freedom in tuning the method and/or its algorithm.

According to an embodiment of the present inventive method, the at least one cardiac vector signal is monitored continuously for a defined period of time, e.g., for 30 minutes, 1 hour, 2 hours, 3 hours, 4 hours, 5 hours, 6 hours, up to 24 hours. For example, the at least one cardiac vector signal is monitored non-stop when the implantable device is active. In an embodiment, extracting the two or more separate features from the at least one cardiac vector signal is performed anytime based on the monitored at least one cardiac vector signal.

According to an embodiment, the feature extraction is performed by a processing unit (PU). The processing unit may be part of an implantable device, or an external device. According to an embodiment, an implantable device comprises said processing unit, and an external device comprises a processing unit.

Moreover, in an example, when the two or more features were extracted from a part of the cardiac vector signal, data of said part of the cardiac signal and/or the extracted features are transmitted to a processing unit (which may be external) for further processing. According to an example, the processing unit performs signal processing by evaluates data of the part of the cardiac vector signal and/or evaluates the extracted features regarding a disturbance of the implantable device, e.g., a defect of the hardware status, a hardware deviation and/or disturbance caused by other (non-physiological) origin. Performing at least a part of the signal processing with an external device having a processing unit instead of the implantable device safes energy for the implant battery. For the signal processing, the external unit may use feature extraction techniques disclosed in the present invention, or other methods for signal processing and signal filtering.

An advantage according to embodiments of the inventive method and system is the high sensitivity and reliability due to the continuous monitoring of at least one cardiac vector signal for the detection of a defect in the hardware status, a hardware deviation and/or disturbance caused by other (non-physiological) origins. A continuous monitoring of the cardiac vector signal ensures fail-safe detection of abnormalities occurring in the cardiac vector signal.

Notably, with the improved detection performance, a faulty hardware status may be detected earlier which may increase the possible time frame for subsequent medical intervention. Hence, the implantable device may not reach a state where it can hurt a patient, and there may be a sufficient time frame for planning an according corrective action (e.g., scheduling a surgery for fixing/exchanging the implantable device, changing the shock path and/or the sensing vector, etc.).

As an example, the derived hardware status may comprise a likelihood of a hardware deviation since the exact hardware status may not be correctly determined. In an example, the classification criteria of a classification group may be set in such a way that they are associated with the likelihood of a hardware deviation. For example, the likelihood of the hardware deviation may thus serve as a figure of merit about the degree of (hardware) perturbation onto the (physiological) cardiac vector signal. The likelihood may be compared to a likelihood threshold wherein further action may be triggered if the likelihood threshold is exceeded (e.g., further analysis/testing may be performed by medical personnel). For example, the likelihood of a hardware deviation may be referred to as pₙₒᵢₛₑ. To this regard a low pₙₒᵢₛₑ (e.g., pₙₒᵢₛₑ<0.5) may indicate a low probability of a hardware deviation, whereas a high pₙₒᵢₛₑ (e.g., pₙₒᵢₛₑ>0.8) may indicate a high probability of a hardware deviation.

For example, the implantable device referred to in the present invention may be a cardiac implant (e.g., a pacemaker, a defibrillator, an implantable cardioverter defibrillator, etc.) which may acquire the at least one cardiac vector signal between at least one pair of electrodes. A electrode may be an electrode of a stimulation lead, or a casing (of the implantable device), or any other electrically conductive terminal of the cardiac implant (or implantable device) in contact with a body tissue. In an example, the electrode may be a transvenous probe implanted into a heart chamber. For example, the at least one cardiac vector signal may be a signal component of an intracardiac electrogram (IEGM) when at least one of the electrodes is positioned within a heart chamber (e.g., a left atrium, a left ventricle, a right atrium, a right ventricle) for sensing its heart activity. In that regard, the extracted one or more features may be referred to as IEGM features.

In another example of the method, at least one of the extracted two or more separate features may be based at least in part on a refractory period. For example, the implantable device may be a cardiac implant (e.g., a pacemaker, a defibrillator, an implantable cardioverter defibrillator, etc.) which may acquire the at least one cardiac vector signal between at least one pair of electrodes during the refractory period of the heart activity. The refractory period may comprise the physiological/biological refractory period (e.g., an effective refractory period—ERP, an absolute refractory period—ARP, etc.), as well as the technical refractory period (e.g., a programmed refractory period associated with the physiological/biological refractory period) associated with the heart activity. Usually, the refractory period may be referred to as the blanking period in which the sensing of events is inhibited and/or ignored by the cardiac implant. This may be due to medical reasons, for example to prevent a stimulation response based on an (irrelevant, e.g., non physiological event/crosstalk caused event) event in the refractory period. Usually, events in the refractory period may not even be visible in most cardiac signals since they are a priori excluded. However, the inventors have surprisingly found out that the events in the refractory period may be highly beneficial for deriving a hardware status (e.g., of the cardiac implant) according to the present invention. For example, at least one of the extracted two or more separate features may be based at least in part on a predetermined number of events in the refractory period. In another example, at least one of the extracted two or more separate features may be based at least in part on an event type in the refractory period (e.g., an event type may be a particular signal pattern/signature, etc.).

It is noted that it may alternatively also be possible to use only a single extracted feature in the methods described herein. This may particularly be advantageous in case the extracted feature is based on a refractory period, as described herein.

In an example, the classification may be based at least in part on one of the following: a decision tree, a random forest, a convolutional network, a support vector machine and/or any other classifier algorithm.

For example, the classification may be implemented by a random forest (i.e., a random forest classifier) comprising a plurality of decision trees. For exemplary purposes a simple decision tree classifier is illustrated in the following. Firstly, two separate cardiac vector signals may be received (e.g., a first cardiac vector signal and a second cardiac vector signal). The first cardiac vector signal may be acquired between a first pair of electrodes by the implantable device, wherein the second cardiac vector signal may be acquired between a second pair of electrodes (e.g., the first and second pair of electrodes may also share one electrode). Subsequently, the method may comprise extracting feature A and feature B from the first cardiac vector signal. In addition, feature C may be extracted from the second cardiac vector signal. The feature extraction may be implemented by any means suitable for a feature extraction (e.g., a suitable feature extraction algorithm). The features A, B, and C may be numerical values whereas exemplary features as generally described herein may be used. Subsequently, the features A, B and C may be inputted to the random tree classifier. In this example the random tree classifier may comprise a first decision tree and a second decision tree. Each decision tree may be defined by a different set of decision nodes regarding the features A, B, and C. A decision node may comprise comparing a numerical value of a feature (e.g., A, B, C) to one or more threshold values (e.g., A′, A″, . . . , B′, B″, . . . , C′, C″, . . . ). For example, the first decision tree may comprise a first decision node that checks if A>A′. In this example if A>A′ is not true, the classification may result in a first leaf node which may be a first classification group defined by the classification criteria A≤A′. However, if the result (A>A′) is true, a second decision node may check if B>B′. The latter result (i.e., true or false) may thus define a second leaf node which may be a second classification group defined by the classification criteria A>A′ and B>B′. Further it may define a third leaf node which may be a third classification group defined by the classification criteria A>A′ and B≤B′.

The second decision tree may comprise a different set of decision nodes with a different set of classification criteria (e.g., C<C′: B>B″), which may span a fourth classification group, a fifth and a sixth classification group. In this example, the features A, B, C may fall into the second classification group associated with a pₙₒᵢₛₑ of 0.7 (defined by the first decision tree) and the fourth classification group associated with a pₙₒᵢₛₑ of 0.5 (defined by the second decision tree), for example. The derived hardware status may be based on averaging the results of the classification groups (e.g., in this case the derived hardware status may be pₙₒᵢₛₑ=(0.7+0.5)/2=0.6).

Notably, common decision tree classifiers are usually more complex as illustrated in this example, as the person skilled in the art may be aware. For example, common random forest classifiers (or ensemble classifiers based on decision trees) may have a higher number of decision trees, a greater depth/complexity of a decision tree as explained, wherein the derived hardware status may also be based on a majority vote (e.g., if a classification group is not a numerical value but a hardware category), etc.

In an example of the method, at least one of the extracted two or more separate features may be based at least in part on a signal transformation of the at least one cardiac vector signal. The signal transformation may be any type of mathematical and/or signal transformation performed onto the at least one cardiac vector signal and/or a plurality of cardiac vector signals. For example, the transformation may be a frequency transformation (e.g., a Fourier transformation, a Laplace transformation, etc.), a mathematical combination of one or more cardiac vector signals (e.g., an auto-correlation, a cross-correlation, an addition of two different cardiac vector signals, a phase plot, etc.), among many other transformations that can be achieved by signal processing. For example, at least one of the extracted two or more separate features may be based at least in part on a frequency associated with the at least one cardiac vector signal. For brevity purposes, it is outlined that any cardiac vector signal mentioned in the present disclosure may also be understood as involving a signal transformation of the cardiac vector signal.

In an example the method may be based at least in part on a predetermined time interval. For example, the method may be adapted to extract the two or more features over a predetermined time segment. The derived hardware status may thus be quantified over the time segment Δt (e.g., between an initial time to and a later time t₁). In an example, the predetermined time interval may be referred to as an episode. The time segment Δt may be chosen to represent a reasonable time frame for deriving the hardware status. The method may further comprise analyzing a plurality of (e.g., consecutive) predetermined time segments for deriving the hardware status. In an example, the plurality of predetermined time segments may comprise the same time intervals Δt. This may be beneficial for computational purposes, as well as deriving the hardware status. For example, the hardware status may be based at least in part on a predetermined proportion of time segments having a particular hardware status (e.g., the hardware status may be based on analyzing three segments, wherein a hardware status may be derived if two of the three segments have a high likelihood of a hardware deviation).

In another example of the method at least one of the extracted two or more separate features may be based at least in part on a complexity of the at least one cardiac vector signal. For example, the complexity of the signal may be derived based on any common complexity algorithm, complexity definition, complexity principle, etc. In an example, the complexity may be derived based on a Lempel-Ziv complexity and/or a Kolmogorov complexity.

In another example of the method at least one of the extracted two or more separate features may be based at least in part on filtering the at least one cardiac vector signal. For example, a filter may be applied to the at least one cardiac vector signal by means of signal processing to create a filtered cardiac vector signal. In one example, at least one of the two or more separate features may be based on the filtered cardiac vector signal. To illustrate an example, filtering as stated herein may be understood as a class of signal processing which results in a complete or partial suppression of some aspect of the signal (e.g., the at least one cardiac vector signal). Thus, the filtering may not be exclusively limited to filters acting in the frequency domain. Notably, the filter may be any filter type common in the field of signal theory (e.g., low pass filter, high pass filter, band pass filter, Tschebyscheff filter, Butterworth filter, etc.). In another example, at least one of the extracted two or more separate features may be based on an output value of a filter (e.g., a VF-Filter leakage (VF: ventricular fibrillation), a filter leakage measure (FLM), etc.). The VF-Filter leakage may be based on the VF Filter Leakage Method as described by Kuo S, Dillman R., Computer Detection of ventricular fibrillation, Computers in Cardiology 1978, IEEE Computer Society Press, 1978:347-9.

In another example of the method at least one of the extracted two or more separate features may be based at least in part on applying a statistical analysis to the at least one cardiac vector signal. For example, the statistical analysis may be based on a number of specific values, a particular distribution (e.g., a probability distribution, a shape of a probability distribution, a signal value distribution, a histogram etc.), a statistical relationship between two random variables, a correlation, a dependence, etc. which may be associated with the at least one cardiac vector signal. In an example of the method at least one of the extracted two or more separate features may be a kurtosis. The kurtosis may be calculated based on a distribution of the signal values. In an example, the kurtosis may be calculated based on the definition by Pearson, whereas no biasing correction may be implemented:

$$\frac{\frac{1}{n} {\sum }_{i = 1}^{n} {( {x}_{i} - \overline{x} )}^{4}}{{( \frac{1}{n} {\sum }_{i = 1}^{n} {( {x}_{i} - \overline{x} )}^{2} )}^{2}}$$

In an example, n may be the number of signal values to be analyzed, xᵢ may be a signal value, x̅ may be an arithmetic mean of all n signal values.

In another example of the method at least one of the extracted two or more separate features may be based at least in part on a heart rate. The heart rate (HR) may be associated with the at least one cardiac vector signal. For example, the heart rate may be determined based on an R-R interval, a P-P interval etc. Further, the method may comprise determining the heart rate (deviation) determined on different cardiac vector signals.

In another example of the method at least one of the extracted two or more separate features may be based at least in part on a plurality of received cardiac vector signals. For example, for determining a particular feature, at least two different (i.e., separate) cardiac vector signals may be necessary. To illustrate an example, it may be necessary to (mathematically) combine two different cardiac vector signals to determine the particular feature. As stated herein, this may be the case when determining a cross-correlation, a sum of cardiac vector signals, a convolution of cardiac vector signals, etc.

In another example of the method at least one of the extracted two or more separate features may be based at least in part on transforming the at least one cardiac vector signal into a binary vector string based at least in part on a binary threshold value. For example, this may enable determining a distribution of the signal values around the binary threshold value for further signal processing. To illustrate an example, the cardiac vector signal may be based on a predetermined time segment, wherein the cardiac vector signal may comprise 5 signal values (e.g., 2 mV, 4 mV, 5 mV, 0 mV, 1 mV in that order). The binary threshold value may be set to be 3 mV. Subsequently, every signal value may be evaluated separately against the binary threshold value (e.g., evaluating if the signal value is greater/smaller than the binary threshold value or equal to the binary threshold value) wherein an evaluation result (e.g., true or false or equal to) may be transformed into a binary number (e.g., 0 or 1) in the order of the signal values. In this example, the cardiac vector signal may result in a binary vector string of 0-1-1-0-0 (e.g., when evaluating if each signal value is greater than the binary threshold of 3 mV and false may be outputted as 0 and true may be outputted as 1). It may also be conceivable in this example that the cardiac vector signal may result in a binary vector string of 1-0-0-1-1 (e.g., when evaluating if the signal value is smaller than the binary threshold of 3 mV and false may be interpreted as 0 and true may be interpreted as 1). The binary vector string may be used for further signal processing and/or feature extraction of the inventive method.

In another example of the method at least one of the extracted two or more separate features may be based at least in part on a phase space of the at least one cardiac vector signal. For example, a feature may be based on a time delay phase plot (e.g., as mentioned in “Amann A, Tratnig R, Unterkofler K. Detecting ventricular fibrillation by time-delay methods. IEEE Trans Biomed Eng. 2007”). The at least one of the extracted two or more separate features may be based on the number of boxes visited in the time delay phase plot, for example.

In an example of the method at least one of the extracted two or more separate features may comprise at least one of the following features associated with the at least one cardiac vector signal: a number of events in a refractory period, a complexity of a binary vector string, a VF-filter leakage, a kurtosis, a heart rate, an R-R interval, a maximum of a signal amplitude, a sum of signal values, a sum of signal values normalized by a maximum of the signal amplitude, a number of samples with a signal amplitude within a certain range after bandpass filtering, a first spectral moment (normalized), a mean of a frequency distribution divided by a reference peak, a maximum of an absolute autocorrelation function, a variance of a binary vector string, a number of transitions from “0” to “1” in a binary vector string, a maximum number of “0”s and/or of “1”s in a binary vector string, a phase space, a proportion of an area covered in a phase space plot, a Pearson correlation coefficient of an absolute autocorrelation function, a proportion of an area contained within a certain frequency range, a fundamental frequency.

In an example for deriving the hardware status, the extracted separate features may comprise the number of events in the refractory period and the kurtosis associated with at least one cardiac vector signal. In another example, the extracted features may further comprise the heart rate in addition, as well.

In another example, the extracted features may comprise the number of events in the refractory period and the kurtosis, and optionally one or more of the heart rate, the maximum of a signal amplitude, and/or the complexity of the binary vector string. Additionally, one or more of the sum of signal values normalized by a maximum of the signal amplitude, a proportion of an area covered in the phase space plot, and/or a number of transitions from “0” to “1” in the binary vector string associated with at least one cardiac vector signal may be comprised.

In another example, the at least two features may be selected from the number of events in the refractory period and the heart rate (e.g., a heart rate determined from an R-R interval) associated with at least one cardiac vector signal, or comprise both.

In another example, the at least two features may be selected from the number of events in the refractory period and the sum of signal values normalized by a maximum of the signal amplitude associated with at least one cardiac vector signal, or comprise both.

In another example, the at least two features may be selected from the number of events in the refractory period and the number of samples with a signal amplitude within a certain range after bandpass filtering associated with at least one cardiac vector signal, or comprise both.

In an example of the method, the deriving of the hardware status may be further based on at least one supplementary feature which is not necessarily associated with a cardiac vector signal acquired by the implantable device between at least one pair of electrodes. The supplementary feature, for example, may not directly fall into a category as outlined herein for the extracted two or more separate features. For example, the supplementary feature may not be associated with an intracardiac electrogram diagram (IEGM) signal for medical purposes. However, in an example, the feature may be based on a vector signal acquired by the implantable device between at least one pair of electrodes for technical reasons, e.g., the feature may be based on a stimulation/pacing threshold, an impedance measurement which requires determining a signal between at least one pair of electrodes. The supplementary feature may further be based on a statistical analysis associated with the technical application of the implant (e.g., a number of charging events, a number of stimulation/pacing shocks). The supplementary feature may be determined by signal processing and/or by reading out data (e.g., logging data, a data history, etc.).

In an example of the method, it may further comprise determining the supplementary feature wherein the supplementary feature may comprise at least one of the following features: a number/proportion of aborted stimulation shocks, a number/proportion of successful stimulation shocks, a number of recorded ventricular extrasystoles, a number of heart activity intervals which are shorter than a typical physiological interval, an excursive change in impedance above a defined impedance threshold, an impedance below a defined impedance threshold, an impedance above a defined impedance threshold, a variability of a feature over a predetermined time span, an absolute occurrence of exceeding/underrunning of a feature threshold in a predetermined time span, a relative occurrence of exceeding/underrunning/crossing of a feature threshold in a predetermined time span.

In an example of the method the derived hardware status may comprise at least one of the following: a likelihood of a hardware deviation, a sustained/intermittent/temporary technical malfunction, an electrode breakage, a technical malfunction due to an external source, an external noise, an electric hum, an interference by a medical equipment, an interference from magnetic resonance imaging. For example, a classification group may be at least one category of the stated hardware statuses.

Some examples described herein may particularly be advantageous for determining an electrode breakage.

In an example, the method may further comprise activating a response, based at least in part on the derived hardware status. For example, the response may comprise sending a notification/alert, recording the cardiac signals on a storage medium, adapting detection parameters of the implantable device, adapting the stimulation output of the implantable device (e.g., a cardiac implant), activating of additional measurements (e.g., impedance measurements), etc.

In an example the deriving of a hardware status may be performed by an artificial intelligence system and/or machine learning system that has been trained with cardiac vector signals acquired by well-functioning and/or malfunctioning implantable devices. Especially in the field of cardiac implants a variety of historical data may be available wherein the data may comprise clinical information, as well as hardware information seen in the cardiac vector signals. In a preferred example, the training data may be based on historical cardiac vector signals which may be associated with a clinical intervention that took place due to a hardware defect.

According to an embodiment, for generating the specific feature vector, analogous front-end filtering and/or data compression is performed.

A second aspect relates to a processing unit for monitoring an implantable device. The processing unit may comprise means for performing the method (and method examples) as outlined herein. In one example, the processing unit may be an external unit not comprised in the implantable device. For example, the processing unit may be comprised in an external computational device residing outside of the patient (e.g., a telemonitoring system), wherein the implantable device may be configured for communicating with the external computational device. This may be beneficial since the (external) processing unit and the external computational device may facilitate calculations with a higher computational complexity without being limited by the implantable device's energy consumption constraints. The (external) processing unit may, for example, be used to routinely check the hardware status based on a historical record of the at least one cardiac vector signal, which may be sent to the (external) processing unit in regular intervals and/or when possible.

According to an embodiment, the historical record is obtained in the last 30 minutes, last hour, last 2 hours, last 5 hours, last 24 hours, last 36 hours, last 48 hours, last 3 days, last 4 days, last 5 days, or last 7 days.

Notably, in an example, the method may comprise extracting a, e.g., one, feature from the at least one cardiac vector signal by signal processing, determining one supplementary feature not necessarily associated with a cardiac (vector) signal, deriving the hardware status of the implantable device based at least in part on the classification of the extracted feature and the supplementary feature.

In another example, the processing unit may be comprised in the implantable device itself. This may enable a runtime evaluation of the hardware status of the implantable device. The method may be adapted to suit the implantable devices specifications regarding the computational effort, storage capabilities and/or energy consumption. This may enable a steady monitoring of the hardware status.

In another example, the processing unit may be comprised in a separate implantable device. In this example, the separate implantable device may be optimized for computational capabilities to perform the methods described herein.

In any case, the (separate) implantable device may be configured for communication with an external device.

A third aspect relates to a system for monitoring an implantable device. The system may comprise an implantable device for acquiring at least one cardiac vector signal. The system may further comprise the processing unit as outlined herein. For example, the system components may be communicatively coupled over a wire or wirelessly. In an example, the system may be configured such that the processing unit may request the at least one cardiac vector signal by the implantable device. In another example, the system may be considered the implantable device comprising the processing unit (as outlined herein). In another example, the system may be considered a processing unit residing outside of the patient (e.g., the processing unit may be comprised by a telemonitoring system) and being communicatively coupled to the implantable device. In another example, the system may be considered as comprising a processing unit comprised in a separate implantable device.

According to an embodiment of the inventive system, the implantable device and/or an external device comprise a processing unit according to the present invention.

According to an embodiment of the inventive system, the implantable device is configured to transmit data of at least a part of the cardiac vector signal, or data of at least a feature extracted from the cardiac vector signal to the external device, wherein the external device is configured to evaluate the data of at least a part of the cardiac vector signal, or data of at least a feature extracted from the cardiac vector signal. According to the embodiment, at least a part of the cardiac vector signal is evaluated by the processing unit of the implantable device in a first step (e.g., by performing feature extraction). Then, the data representing at least a part of the cardiac vector signal or data representing the evaluated cardiac vector signal is transmitted from the implantable device to the external device in a second step. In a third step, the data is further evaluated by the processing unit of the external device, e.g., by using a method according to the present invention, or by performing another signal evaluation process. Due to that workflow, battery life of the implant can be saved.

According to embodiments of the present invention, the external device may be a clinician programmer, a handheld device (e.g., tablet or smartphone), or an external server system.

A fourth aspect relates to a computer program. The computer may comprise instructions to perform a method (or method examples) as outlined herein, when the instructions are executed by the processing unit as outlined herein and/or the processing unit of the system as outlined herein.

It is noted that the method steps as described herein may include all aspects described herein, even if not expressly described as method steps but rather with reference to an apparatus (or device or system). Moreover, the devices as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following, the Figures of the present disclosure are listed:

FIG. 1 Schematic representation of an exemplary embodiment of a processing unit and a system comprising an implantable device having the processing unit according to the present invention.

FIGS. 2a-2b Schematic representation of an exemplary embodiment of a method according to the present invention. FIG. 2a schematically represents a feature extraction out of cardiac vector signals and a feature classification by a classifier. FIG. 2b schematically represents a decision tree used in the classifier.

FIG. 3 Schematic representation of an exemplary embodiment of a method according to the present invention for triggering a response depending on a derived hardware status.

FIG. 4 Block diagram showing an exemplary embodiment of a method according to the present invention using features of a cardiac vector signal and feature not associated with a cardiac vector signal.

FIG. 5 Schematic representation of an exemplary embodiment of a system comprising an implantable device and a processing unit PU according to the present invention.

FIGS. 6a-6b Schematic representation of exemplary embodiments of implantable devices comprised in the system according to the present invention. FIG. 6a represents a dual coil implantable device. FIG. 6b represents a multi-electrode implantable device.

FIGS. 7a-7b Schematic representation of an exemplary embodiment of a method according to the present invention. FIG. 7a schematically represents a feature extraction out of cardiac vector signals and a feature classification by a classifier. FIG. 7b schematically represents a decision tree used in the classifier.

FIGS. 8a-8b Schematic representation of a display component used in a system according to the present invention. FIG. 8a represents a first example of the display component. FIG. 8b represents further examples of the display component.

### DETAILED DESCRIPTION

Subsequently, presently preferred embodiments will be outlined, primarily with reference to the above Figures. It is noted that further embodiments are possible, and the below explanations are provided by way of example only, without limitation.

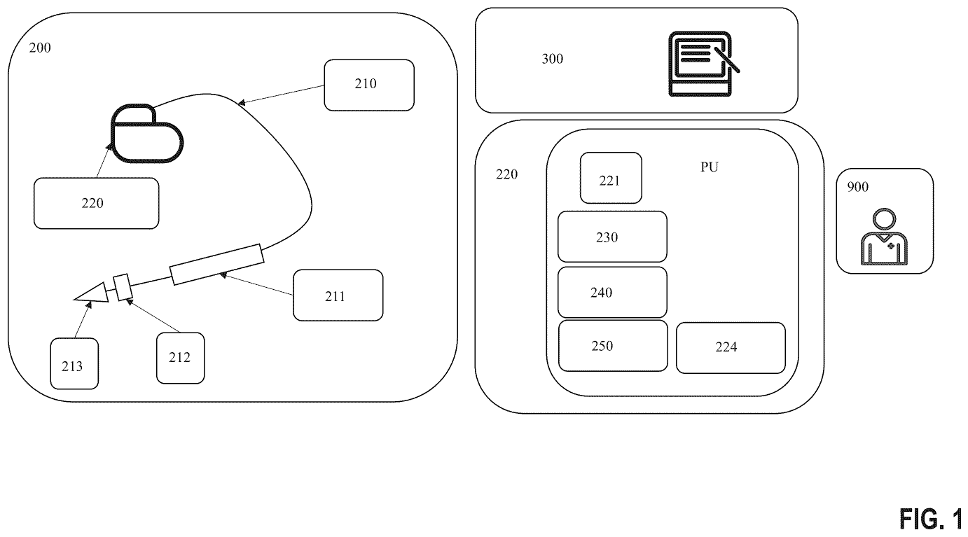

FIG. 1 shows a schematic representation an exemplary embodiment of a processing unit and a system according to the present invention. The system may comprise the implantable device 200 comprising the processing unit PU. The system may further comprise an external programming unit 300. The programming unit 300 and the implantable device 200 may be communicatively coupled (e.g., by wireless communication). The external programming unit 300 may be a component for requesting and displaying stored data and alerts determined by the implantable device 200. Further, the external programming unit 300 may be used to program and/or adapt settings of the implantable device 200 (e.g., of its feature extraction unit 230, classification unit 240, control unit 250, as outlined herein). The implantable device 200 and the inventive system may be adapted to be used by a clinical user 900. In an example, the processing unit PU and/or the implantable device may further comprise at least one of the following: an analog filter, a digital filter, a means for offset compensation, a signal processor.

The implantable device 200 comprising the inventive processing unit PU may be a cardiac implant which is configured for implanting into a patient. For example, the implantable device 200 may be a pacemaker (e.g., lead-based, leadless, etc.), a defibrillator, a cardioverter defibrillator, etc. The implantable device 200 may comprise a right ventricle (RV) electrode lead 210, a (RV) shock-coil electrode 211, a (RV) ring electrode 212, and/or a (RV) tip electrode 213. The implantable device may further comprise a main unit 220. The implantable device 200 may be configured to stimulate a heart chamber and/or a human body by electrical stimulation (i.e., electrical shock) over its electrodes (e.g., over the shock-coil electrode 211, the ring electrode 212, and/or the tip electrode 213). The implantable device 200 may further be configured to determine a cardiac vector signal by sensing electrical signals in the body (e.g., sensing heart activities in a heart chamber). The cardiac vector signal may be determined between a pair of (sensing) terminals which are in contact with a body tissue. In an example, the terminals may be the electrodes (211, 212, 213), as well as the casing of the active implant component 220. For example, a first cardiac vector signal may be defined between the ring electrode 212 and the tip electrode 213. A second cardiac vector signal may be defined between the shock-coil electrode 211 and the ring electrode 212, etc. However, any terminal combination may be used for defining a cardiac vector signal regarding the implantable devices as outlined herein.

The main unit 220 of the implantable device 200 may comprise the processing unit PU according to the present invention. The processing unit PU may comprise an Analog-to-Digital converter 221 (i.e., an AD-converter). The AD-converter 221 may be coupled to an interface for receiving at least one cardiac vector signal determined by the implantable device. The AD-converter 221 may be used to convert the analog cardiac vector signal into a digital cardiac vector signal which may be used for further signal processing. The processing unit PU may further comprise a feature extraction unit 230 for extracting features out of the determined cardiac vector signals. The feature extraction unit 230 may be implemented by way of hardware and/or software (e.g., an ASIC, a computer algorithm running on a processor, a microcontroller etc.). The main unit 220 may further comprise a classification unit 240. The classification unit 240 may be implemented by ways of hardware and/or software (e.g., an ASIC, a computer algorithm running on a processor, a microcontroller etc.). The classification unit 240 may implement a classifier which executes a classification method for classifying the extracted features. The processing unit PU may further comprise a storage unit 224 comprising a storage medium for storing data. The processing unit PU may further comprise a control unit 250. The control unit may be configured for activating a response based on a classification of the extracted features. The control unit 250 may be implemented by ways of hardware and/or software (e.g., an ASIC, a computer algorithm running on a processor, a microcontroller etc.). The control unit 250 may be configured to couple the AD-converter 221, the feature extraction unit 230, the classification unit 240 and/or the storage unit 224 with each other in a controlled way. For example, the control unit 250 may facilitate the data exchange between the units (e.g., by hardware and/or software). In addition, the control unit 250 may be configured for triggering data storage associated with the feature extraction unit 230 and/or the classification unit 240. In other embodiments, the described functionalities may be implemented differently, e.g., feature extraction unit 230, classification unit 240 and/or control unit 250 may be implemented in a single unit.

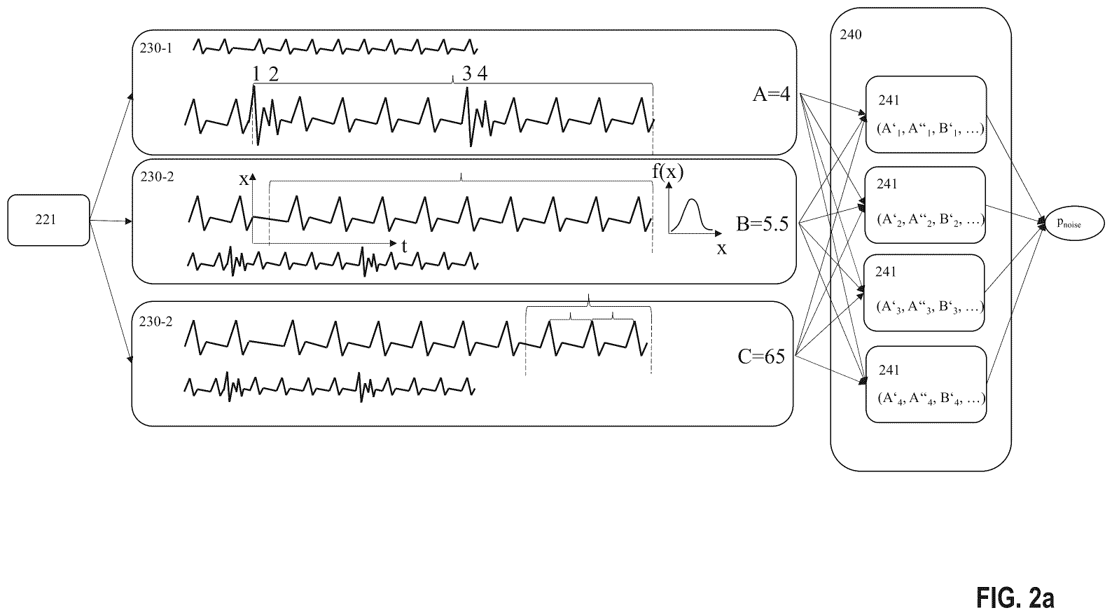

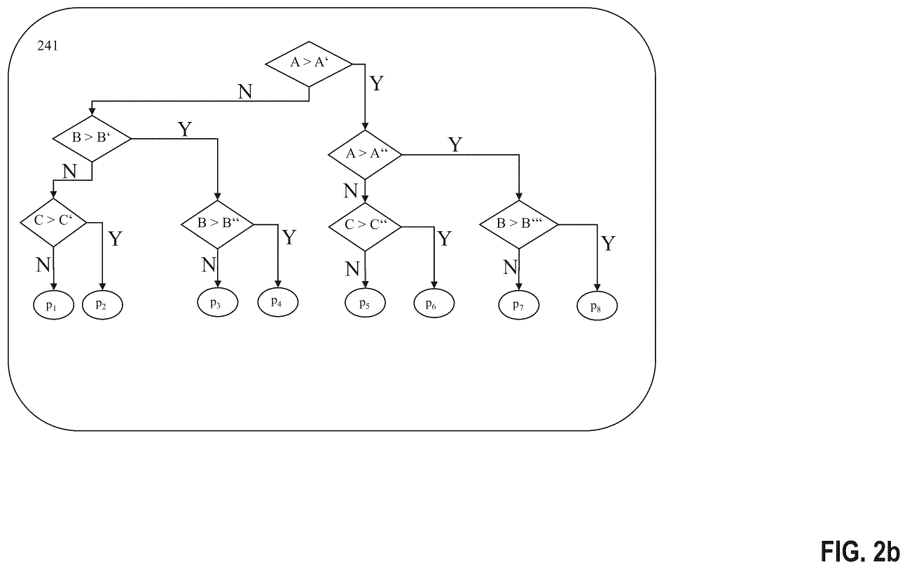

FIGS. 2a/b schematically represent an exemplary embodiment of a method according to the present invention. FIG. 2a shows a feature extraction out of cardiac vector signals which may be inputted into a classifier for a classification result. In this example, the AD-converter 221 may have received two cardiac vector signals. The AD-converter 221 may subsequently create two corresponding digital cardiac vector signals which may be communicated to the feature extraction unit 230. In the example of FIG. 2a/b, a first cardiac vector signal is depicted in 230-1 and second cardiac vector signal is shown in 230-2, wherein the first cardiac vector signal in 230-1 is defined and recorded between the ring electrode 212 and the tip electrode 213. In 230-1 the number of refractory senses (1-4) is counted in the first cardiac vector signal. Further in the example, the second cardiac vector signal in 230-2 is defined and recorded between the shock-coil electrode 211 and the ring electrode 212. In 230-2, the kurtosis and heart rate of the second cardiac vector signal are determined for the cardiac vector signal. Subsequently, the feature extraction unit 230 may determine a particular set of features. The number of extracted features may be chosen to be two, three or four, for example, when the processing unit PU is comprised in the implantable device 200. In the example of FIG. 2a three features A, B, C associated with the (digital) cardiac vector signals may be extracted. To this regard feature A may be the number of events in a refractory period of the first cardiac vector signal 230-1 wherein the events are marked as 1, 2, 3, 4 in the first cardiac vector signal 230-1. Thus, in this example feature A equals 4. Feature B shown in FIG. 2a may be the kurtosis of the second cardiac vector signal 230-2. The kurtosis may be determined by the definition of Pearson, wherein each digital value (e.g., xᵢ) of the second digital cardiac vector signal 230-2 over a certain time period may be used for determining the kurtosis described herein. For exemplary purposes, a probability distribution f(x) of a variable associated with the second cardiac vector signal 230-2 is plotted in FIG. 2a. The kurtosis (and thus feature B) of the exemplary distribution f (x) may be 5.5 in that example. Another feature shown in FIG. 2a may be a heart rate which may be associated with the second cardiac vector signal 230-2. In this example, the determined heart rate may be 65 (bpm). The heart rate may be determined over a specific time interval and/or based on a certain number of peaks of the cardiac vector signal (e.g., R-R intervals). To summarize, in this example the feature extraction unit may have determined that feature A equals 4, feature B equals 5.5 and feature C equals 65. These feature characteristics may be subsequently inputted to the classifier of the classification unit 240. In an example, the classification unit 240 may implement a random forest classifier (although other classifiers may be conceivable such as a convolutional network, a support vector machine, etc.). The random forest classifier may comprise various decision trees 241. In this example, the random forest classifier comprises four decision trees. However, when the processing unit is comprised inside the implantable device 200 up to ten decision trees may be comprised in the random forest classifier. This may meet the requirements for the limited calculation capacity while still guaranteeing a satisfactory sensitivity for deriving a hardware status. Each decision tree may be defined by a certain set of feature thresholds, for example a first decision tree may comprise the feature thresholds of A₁′, A₁″, B₁′, . . . , whereas a second decision tree may comprise the feature thresholds of A₂′, A₂″, B₂′, . . . , and so on regarding the further decision trees. In addition, the decision trees may differ in tree depth (i.e., the amount of checks in each tree). The classifier of the classification unit 240 may output a classification group associated with the feature set A, B, C (i.e., the feature characteristics) that was inputted to the classifier. In this example, the classification group may be referred to as pₙₒᵢₛₑ. Pₙₒᵢₛₑ may be a likelihood of a hardware deviation of the implantable device, e.g., pₙₒᵢₛₑ=0.7 may represent a likelihood of 70% that a hardware deviation may be present. The likelihood of a hardware deviation may comprise the likelihood of an electrode breakage, an intermittent technical malfunction, an external influence, an irregular external influence, an external noise, an electric hum, an interference by a medical equipment, an interference from magnetic resonance imaging. In another example a discrete classification group (i.e., category) may be output instead of pₙₒᵢₛₑ by the classifier (e.g., “electrode breakage”, “external influence”, “external noise”, “low probability of hardware deviation”, “high probability of hardware deviation”, “technical malfunction of electrode system”, technical malfunction external source”, etc.). In this case the decision tree and its classification groups may be accordingly adjusted.

FIG. 2b schematically represents an exemplary decision tree 241 used in the classifier. The decision tree of FIG. 2b may comprise the feature thresholds A′, A″, B′, B″, B′″, C′, C″. It may further comprise eight classification groups of pₙₒᵢₛₑ defined by the leaves of the decision tree. In this case pₙₒᵢₛₑ may be p₁, p₂, p₃, p₄, p₅, p₆, p₇, p₈ depending on the feature characteristics of the features A, B, and C. As outlined in FIG. 2b, the decision tree may start with an initial node that checks whether A>A′. If this initial check is true Y then the subsequent node may check if A>A″. If this initial check is not true N then the subsequent node may check if B>B′. As seen in FIG. 2b further checks may be implemented along the nodes and branches of the decision tree until the inputted features A, B and C fall into one of the classification groups (p₁, p₂, p₃, p₄, p₅, p₆, p₇, p₈). In each decision tree each feature may be checked multiple times with different threshold values. Further, not every feature may have to be used in every available decision tree. The threshold values for the number of events in the refractory period (feature A) may be in the range of 0.06 and 9.7. The threshold values for the kurtosis (feature B) may be in the range of 1.3 and 55. The threshold values for the heart rate (feature C) may be in the range of 22 and 185. To illustrate an example, the first decision tree may have classified the features (A, B, C) into the classification group p₁. A second decision tree of the classifier may have classified the features (A, B, C) into the classification group pₓ. A third decision tree of the classifier may have classified the features (A, B, C) into the classification group p_{y}. A fourth decision tree of the classifier may have classified the features (A, B, C) into the classification group p_{z}. Subsequently, the classifier may average the result of all decision trees in terms of an ensemble-classifier to output the overall classification result pₙₒᵢₛₑ. For example, if p₁=0.5; pₓ=0.6; p_{y}=0.8; pz=0.7 this may lead to a pₙₒᵢₛₑ=(0.5+0.6+0.8+0.7)/4=0.65.

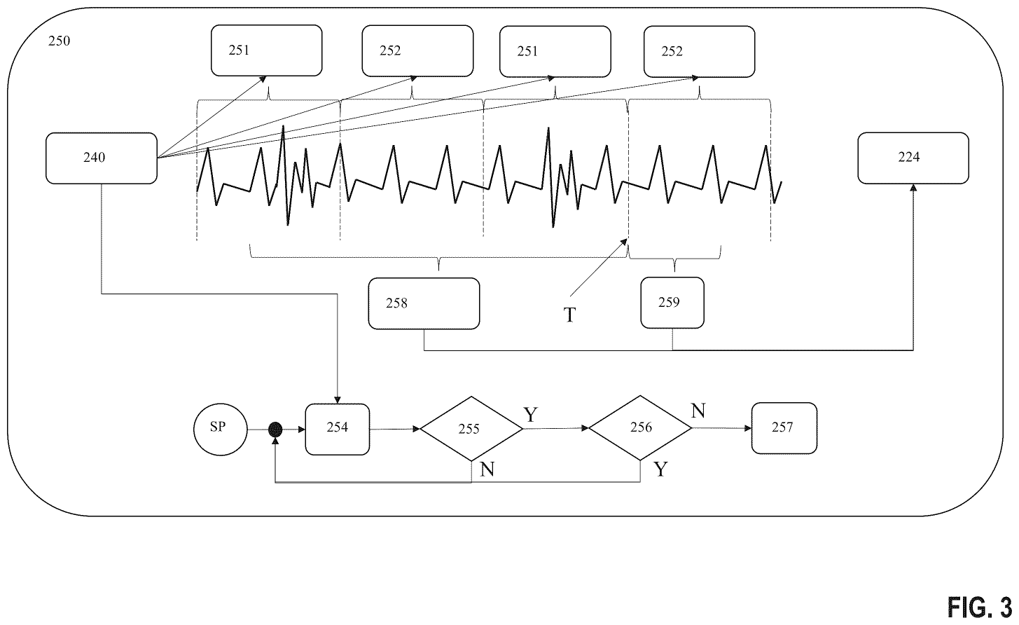

FIG. 3 shows a schematic representation of an exemplary embodiment of a method according to the present invention for triggering a response depending on a derived hardware status. For example, based on the classification result of the classifier (i.e., analysis result) the cardiac vector signals may be stored. In this example, the hardware status may be classified over a predetermined time segment Δt (i.e., a segment length) wherein a plurality of consecutive time segments may be analyzed. Each time segment may be associated with a suspicious hardware status 251 or an unsuspicious hardware status 252 by the classification unit 240. The classification may be initiated at a starting point SP by the control unit 250. Subsequently, the method may comprise continuously applying 254 the classifier of the classification unit 240 onto the cardiac vector signals. To this regard, the classification unit 240) may continuously feedback the classification results of a time segment to the control unit 250. Subsequently, the method may comprise checking 255 if a certain proportion of time segments had a suspicious hardware status (which was derived based on the classification result). This may also comprise checking if X out of Y consecutive segments have had a suspicious hardware status (e.g., if two out of the last three consecutive segments had a suspicious hardware status). If the result is no N the control unit 250 may continue checking 255 if a certain proportion of time segments had a suspicious hardware status. However, if the result is yes Y, a trigger event T may be activated. Subsequently, the method may further comprise checking 256 if a signal recording block is present. If the result is no N, the cardiac vector signal (or the plurality of cardiac vector signals) may be stored in a recording sequence spanning over a time range before and after the trigger event T. In this example, if the trigger event T is activated the last three time segments, as well as the time segment following the trigger event T may be stored. The method may thus comprise storing 257 of the signal segment 258 and 259 with the respective segment length(s), confidence information and one or more details of the classification on the storage unit 224. For example, a signal recording block may be present if a recording sequence due to the same classification results of the trigger event T has already occurred. For example, a trigger event T (associated with a specific classification result) may initialize the storing of a recording sequence. After a defined time span T3 has passed, the same trigger event T (associated with the specific classification result) may not cause the storing of a recording sequence. In this case, the signal block may be removed after a time T4>T3 has passed. This approach may reduce the amount of data that needs to be stored.

In another example, each time segment may be associated with additional information, for example a segment length (defined by a time length Δt of the time segment), a confidence information, a detail of the classification. The confidence information may be based on the analysis of the following features of the classifier of the classification unit 240: a history of cardiac vector assessments by the classifier (e.g., a clustered occurrence of as type X classified episodes may increase the likelihood that the scenario X is real and not a measurement outlier), by comparing the pₙₒᵢₛₑ value of an episode with threshold values (e.g., a low confidence may associated with a low pₙₒᵢₛₑ<0.5; a medium confidence may be associated with a medium pₙₒᵢₛₑ between 0.5 and 0.8; a high confidence may be associated with a high pₙₒᵢₛₑ>0.8).

In another example, after an error was detected by the control unit 250 and/or the implantable device 200 (e.g., a specific event, a specific classification result, a specific episode) the control unit 250 may trigger a confirmation phase for the classification “technical error external source”. In another example, after the classification result “technical malfunction of electrode system” which may be derived as a hardware status the control unit 250 may adapt (with a programmable delay) counter detection criteria for VT (ventricular tachycardia) therapy zones to a programmable alternative value (e.g., higher than the initial values). It may also be conceivable in that example, that the control unit 250 may increase a VF (ventricular fibrillation) detection criteria to a programmable alternative value (e.g., a value higher than the initial value).

In a further example, the processing unit PU may be configured to compare at least two features, wherein the features are from the same category (e.g., the same features, e.g., a heart rate) but extracted from different pairs of electrodes.

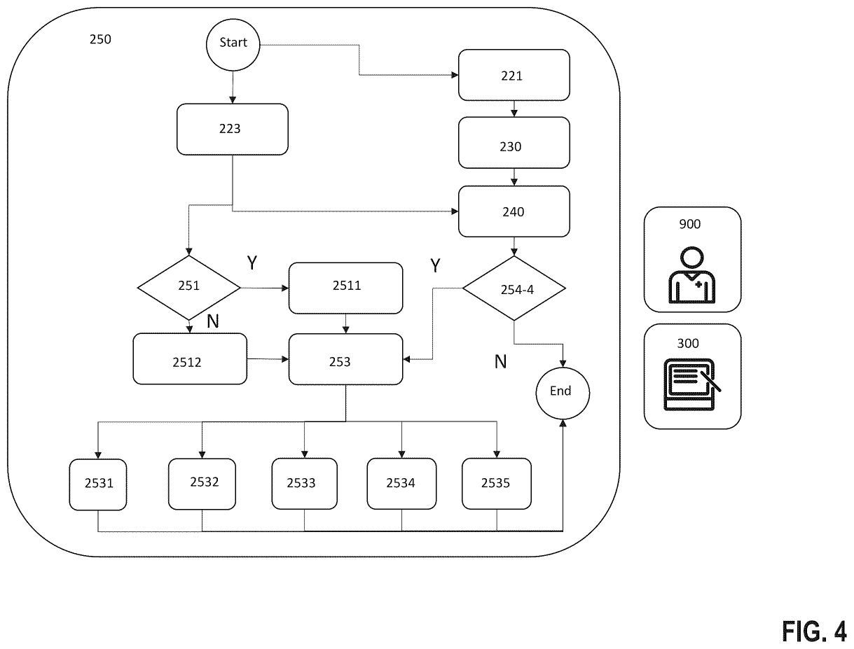

FIG. 4 represents a block diagram showing an exemplary embodiment of a method according to the present invention using features of a cardiac vector signal and features not associated with a cardiac vector signal. In particular, the exemplary method may be executed and/or controlled by the control unit 250. The feature input to the classification unit 240 may not be limited to the features extracted by the feature extraction unit 230 from the at least one cardiac vector signal (provided by the AD-converter 221). In addition, supplementary features 223 may be inputted to the classification unit 240 as well. The supplementary features 223 may not be based on any cardiac vector signal (e.g., they may be based on a timing-based criteria of a heart activity, an impedance from an impedance measurement between at least two terminals of the implantable device, a sensing amplitude test, a capture threshold, etc.). The classification result of the classification unit 240 (e.g., pₙₒᵢₛₑ) may be compared against a feature threshold S1 in an evaluation step 254-4. S1 may be a parameter in a range of 50%-90%. If no suspicious pₙₒᵢₛₑ level is determined (e.g., pₙₒᵢₛₑ<50%) the procedure may be repeated in a following interval. However, if a suspicious pₙₒᵢₛₑ level is determined (e.g., pₙₒᵢₛₑ>50%) this information may be transmitted to a control unit for responsive actions 253. The control unit for responsive actions 253 may, depending on its settings, trigger one or more responsive actions based on the evaluation step 254-4. The settings may be defined by a user 900 via the external programming unit 300 as outlined herein.

The control unit for responsive actions 253 may further evaluate various characteristics before triggering a responsive action. The evaluations of the control unit for responsive actions 253 may comprise: determining a time frame of consecutive segments, in which the classification unit 240 has detected suspicious hardware statuses (e.g., pₙₒᵢₛₑ>S1); determining an occurrence of suspicious time segments defined by X of the last Y time segments being suspicious: determining that at least one different feature independent from the features extracted from the at least one cardiac vector signal is present and/or suspicious (e.g., a suspicious characteristic at the impedance measurement, the pacing threshold test, the sensing amplitude test, etc.): determining that a limiting condition for responsive actions is not violated (e.g., a current/energy budget for further measurements, the occurrence of activating an alert as a response over a predetermined time interval (e.g., only one alert may be activated between two follow-up appointments)).

To illustrate a few example, the responsive actions triggered by the control unit for responsive actions 253 may be: storing 2531 an episode: activating 2532 an alert, wherein the alert may be send with the next telemonitoring message to a telemonitoring system as outlined herein (optionally the alert may comprise a recorded episode) and/or the alert may be displayed in the next interrogation with the external programming unit 300 to the user 900: adapting 2533 the tachycardia detection parameter (e.g., to increase the counter detection criteria relative to a current value and/or to increase the counter detection criteria by a user 900 defined value); deactivating 2534 a shock-therapy-output (or restricting the shock-therapy-output to the highest programmable therapy zone): activating 2535 additional measurements and/or increasing the measurement sequence (e.g., reducing the measurement period for the impedance measurement monitoring associated with one or more cardiac electrodes).

The supplementary features 223 may be parallelly checked in a supplementary evaluation 251 (e.g., not specifically related to the classifier). If one or more of these features is suspicious the control unit for responsive actions 253 may be automatically adjusted regarding its settings/parameters. For example, this may take the form of increasing 2511 the execution probability of a responsive action. However, if no supplementary feature 223 is suspicious, this may result in resetting 2512 the execution probability of a responsive action to an initial value. Further, requirements for increasing or decreasing the execution probability of a responsive action may be conceivable which may enable a hysteresis of the control mechanism.

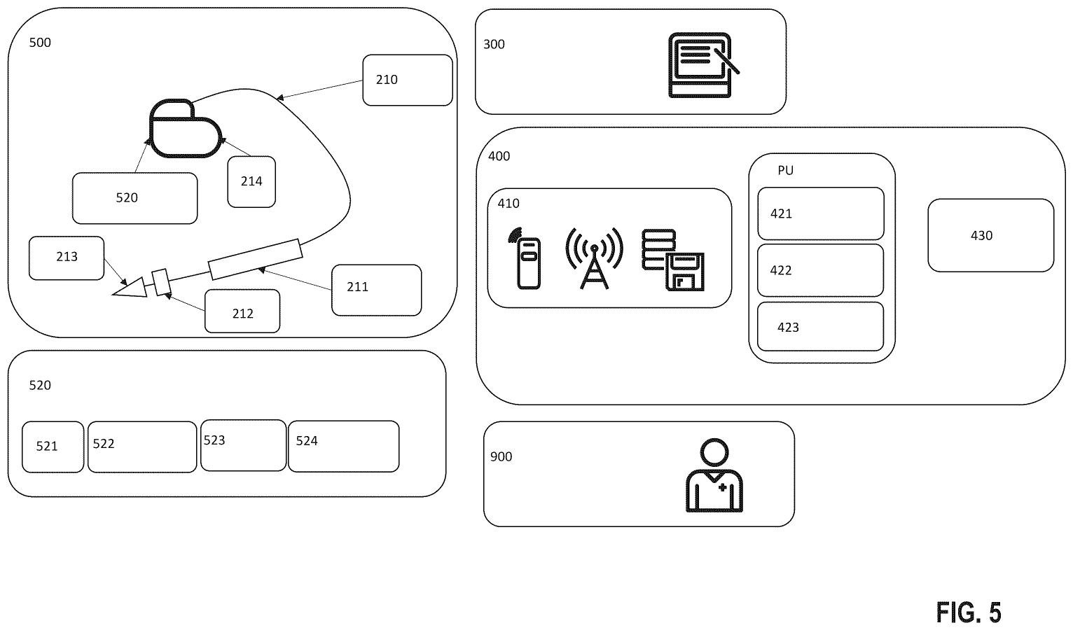

In conclusion, FIGS. 1 to 4 laid out examples in which the processing unit PU that carries out the method for monitoring the implantable device is comprised in the implantable device. However, the inventive concept may not be limited to said example. FIGS. 5 to 8b lay out examples in which the processing unit PU may not be comprised in the implantable device.

FIG. 5 shows a schematic representation of an exemplary embodiment of a system comprising an implantable device and a processing unit PU according to the present invention.

In an example, the implantable device 500 of the system may share parts associated with the implantable device 200 of FIG. 1. In this example, as seen in FIG. 5. The electrode/lead system of the implantable device 500 may be identical to the implantable device 200 (e.g., with the lead 210, the shock-coil 211, the ring electrode 212, the tip coil 213). A first cardiac vector signal may be determined between the shock-coil 211 and the casing of the main unit 520 of the implantable device 500. A second cardiac vector signal may be determined between the ring electrode 212 and the tip coil 213, etc. However, different lead configurations, a different electrode configuration, a different number of leads and/or cardiac vector signals may be conceivable. As indicated in FIG. 5 the implantable device 500 may comprise the main unit 520. The main unit 520 may have the same hardware components, share hardware components and/or have different hardware components as the main unit 220 (of the implantable device 200 in FIG. 1). The main unit 520 may comprise an AD-converter 521, a control unit 522, a storage unit 523, a communication unit 524. The determined cardiac vector signals may be routed through the AD-converter 521. Hence, an analog cardiac vector signal may be transformed into a digital (e.g., discrete) cardiac vector signal. The control unit 522 may trigger that the at least one cardiac vector signal (e.g., the first/second cardiac vector signal) is recorded. The cardiac vector signal may be stored in the storage unit 523. The storage unit 523 may comprise any suitable storage media for storing data. Subsequently, the (stored) data may be communicated over the communication unit 524 to an external device. The communication unit 524 may be configured for sending and/or receiving data when in communication with an external device. The system may further comprise a telemonitoring system 400 which may function as the external device. The telemonitoring system 400 may comprise a communication system 410. The communication unit 524 may transmit the (stored) data (e.g., the cardiac vector signals) to the communication system 410 of the telemonitoring system 400. The telemonitoring system 400 may further comprise a processing unit PU. In this example, the processing unit PU may comprise a feature extraction unit 421, a classification unit 422, and/or a PU control unit 423. To that regard, the processing unit PU may also be referred to as an analyzing unit. The processing unit may analyze the data transmitted from the implantable device 400 by a feature extraction over the feature extraction unit 421, a classification of the features by the classification unit 422 as outlined herein. The PU control unit 423 may trigger responsive actions depending on the classification result as outlined herein. A responsive action may be updating a display component 430 of the telemonitoring system 400. The display component may comprise display hardware, a user interface to show various data on a visual screen for a user. For example, the display component 430 may comprise a monitor which indicates over various windows information associated with the cardiac vector signals and/or classification results. The system may further comprise an external programming unit 300 which may be identical to the external programming unit 300 of FIG. 1. The external programming unit may be configured for requesting stored data on the implantable device 500 (and/or the telemonitoring system) and/or for configuring parameters of the PU control unit 423 and/or the classification unit 422.

In another example, the main unit 520 may comprise the processing unit PU as described herein (e.g., for FIGS. 1-4). To this regard, the method according to the present invention may be used for monitoring the same implantable device over a processing unit PU comprised in the implantable device and/or over a processing unit PU comprised in the telemonitoring system as explained.

In an example, the processing unit PU, the telemonitoring system 400 and/or the implantable device 500 may comprise a data compression unit for compressing data.

In another example, the control unit 522 comprised in the implantable device may be configured to store the following trigger events, heart signals, and/or additional information as episodes: information associated with a tachycardia detection (e.g., information associated with VT—ventricular tachycardia, VF—ventricular fibrillation, SVT—supra ventricular tachycardia, AT—atrial tachycardia, AF—atrial fibrillation), non-persistent tachycardia, a technical trigger (e.g., unexpected event at a pacing/stimulation threshold test, at a sense-amplitude test, at an impedance measurement, an end of persisting pacing), a periodical reference episode.

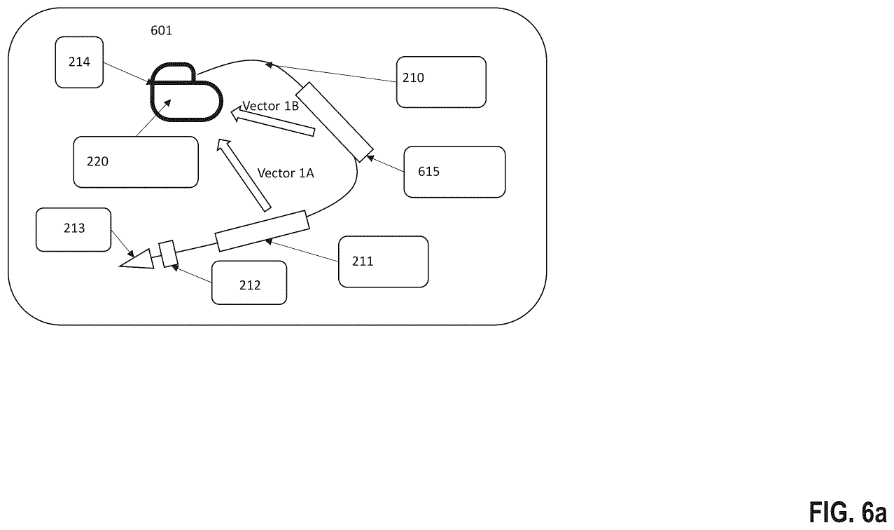

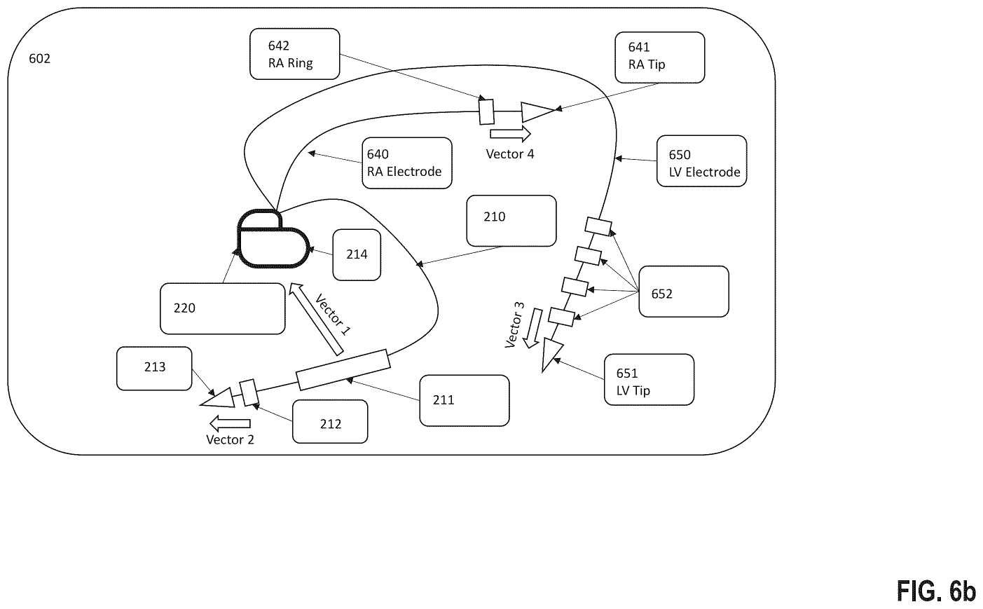

FIG. 6a illustrates an example of an implantable device 601 wherein a cardiac vector signal may be defined by a parallel circuit (i.e., parallel connection) of electrodes. The lead system may additionally comprise an SVC (superior vena cava) shock-coil 615 with regard to the implantable device 500. In this example, a first cardiac vector signal may be determined/defined by the shock-coil 211 and the SVC shock-coil 615 with respect to the casing of the implantable device 601. Hence, this generates a cardiac vector signal defined by the parallelly connected vector 1A and 1B, as illustrated in FIG. 6a. In another example, the first cardiac vector signal may be determined between shock-coil 211 and the casing of the implantable device 601. In another example, the first cardiac vector signal may be determined between the SVC shock-coil 615 and the casing of the implantable device 601.

FIG. 6b illustrates an example of an implantable device 602 which may be used in multiple chambers of the heart (e.g., for pacing/sensing in various chambers, such as left/right ventricle, left/right atrium, etc.). Hence, the electrode lead system may additionally comprise (with respect to the implantable device of FIG. 5) further components. For example, the implantable device 602 may comprise a right atrium (RA) electrode lead 640, comprising a (RA) ring electrode 642 and a RA tip electrode. The implantable device 601 may further comprise a left ventricle (LV) electrode lead 650, comprising one or more (LV) ring electrodes (i.e., ring poles) and a (LV) tip electrode. This example of an implantable device 602, may span various cardiac vector signal which may be determined/defined by the various electrodes as outlined for the implantable device 602. A first cardiac vector signal (e.g., Vector 1) may be defined between the shock coil 211 of the right ventricle (RV) lead and the casing 214 of the implantable device 602. A second cardiac vector signal (e.g., Vector 2) may be defined between the (RV) ring electrode 212 and the (RV) tip electrode 213. A third cardiac vector signal (e.g., Vector 3) may be defined between one of the one or more LV ring electrodes 652 and the (LV) tip electrode. A fourth cardiac vector signal (e.g., Vector 4) may be defined between the (RA) ring electrode 642 and the (RA) tip electrode 641.

It is noted that the additional components outlined with reference to FIGS. 6a and 6b may also be provided for implantable device 200 as described with reference to FIG. 1.

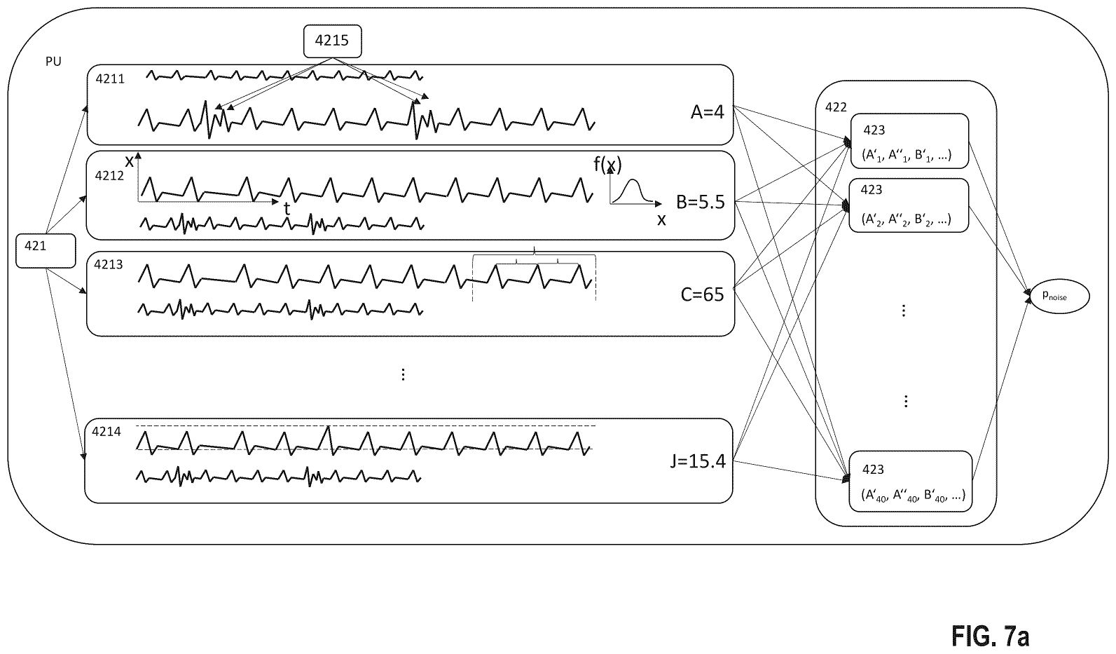

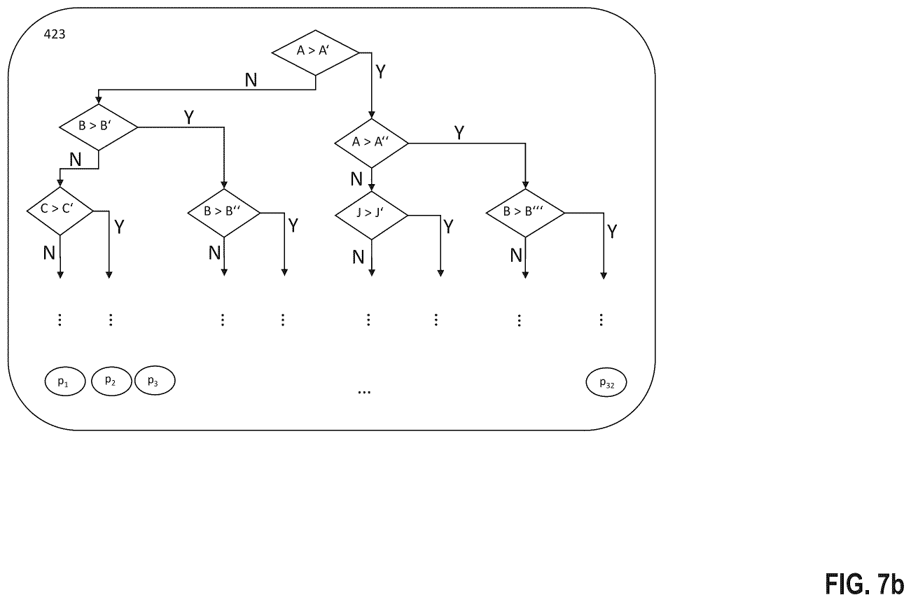

FIG. 7a shows a schematic representation of an exemplary embodiment of a method according to the present invention. FIG. 7a schematically represents a feature extraction out of cardiac vector signals and a feature classification by a classifier. The method may be implemented by the PU comprised in the telemonitoring system 400 (as outlined for FIG. 5). In this example, the classification is based on two cardiac vector signals (i.e., a first and second cardiac vector signal). The cardiac vector signals may be intracardiac electrogram (IEGM) signals acquired over various electrodes residing within a heart chamber. In this example, ten features may be extracted from the two cardiac vector signals (e.g., IEGM signals) acquired by the implantable device (e.g., the implantable device 200, 500, 601, 602) and transmitted to the communication system 410 from the communication unit 524 of the implantable device. The processing unit PU may function in principle as explained for FIG. 1-4 with regards to the feature extraction and classification. The feature extraction unit 421, and the classification unit 422 may be defined by the same hardware/software as the feature extraction unit 230 and classification unit 240. However, when the processing unit is comprised in the telemonitoring system 400 different hardware/software implementations of the processing unit PU may also be conceivable. In particular, the processing unit PU may not be limited by the constraints resulting from the hardware/software requirement in an implantable device (e.g., regarding computational complexity, energy consumption, etc.).

In this example, the feature extraction unit 421 may extract ten features of the two cardiac vector signals. To this regard feature A may be the number of events in a refractory period of the first cardiac vector signal 4211 wherein the events are marked as 4215 in the first cardiac vector signal 4211. Thus, in this example feature A equals 4. Feature B shown in FIG. 7a may be the kurtosis of the second cardiac vector signal 4212. The kurtosis may be determined by the definition of Pearson, wherein each digital value (e.g., xᵢ) of the second digital cardiac vector signal 4212 associated with a time t over a certain time period may be used for determining the kurtosis described herein. For exemplary purposes, a (probability) distribution f(x) of a variable (e.g., a signal amplitude) associated with the second cardiac vector signal 4212 is plotted in FIG. 7a. The kurtosis (and thus feature B) of the exemplary distribution f (x) may be 5.5 in that example. Feature C shown in FIG. 7a may be a heart rate which may be associated with the second cardiac vector signal labeled 4213. In this example, the determined heart rate may be determined for the last three seconds which in this example may be a heart rate of 65 bpm. Feature J shown in FIG. 7a may be a maximum amplitude of a signal value of the second cardiac vector signal (labeled separately as 4214). In this example the maximum amplitude over the time range may be 15.4 (a.u.). The further extracted features may comprise the following: the complexity of the second cardiac vector signal, the sum of the first cardiac vector signal values of the last three seconds normalized by the maximum of the signal amplitude in that time frame, the (VF-filter) leakage of the first cardiac vector signal, a proportion of an area covered in a phase space plot based on a time delay of the second cardiac vector signal, the heart rate of the second cardiac vector signal of the last three seconds and/or the number of “0” to “1” transitions of a binary vector string associated with the second cardiac vector signal. Notably, the number of chosen features may be in the range of 10 to 40. To that regard other features may be chosen as outlined herein.

Subsequently, the numerical values of the features may be routed to the classifier of the classification unit 422. In this example, the classifier may be a random tree classifier comprising various decision trees 423. For example, the random tree classifier may comprise 40 to 200 decision trees 423.

Notably, as seen in this example, when the processing unit PU is comprised in a computational entity (e.g., the telemonitoring system 400) outside of the implantable device the complexity of the classifier and the feature extraction may not be as limited as may be the case when the processing unit is comprised in an implantable device (c.f. explanations to FIGS. 1-4). To illustrate another example, the processing unit PU may be implemented by or comprised in one or more computing devices allowing complex computations which may be the telemonitoring system 400 but also a desktop PC, a server, a cloud-based network service, a computer cluster etc. Without the limitations of the implantable device the decision trees 423 may have a significantly higher number of feature thresholds (A′, A″, B′, . . . , etc.), as well as decision tree depths (i.e., the number of checks in a decision tree). Subsequently, the classifier may average the result of all decision trees in terms of an ensemble-classifier to output the overall classification result (e.g., pₙₒᵢₛₑ).

FIG. 7b shows an exemplary decision tree. The mechanism of the decision tree regarding the extracted features and the classification results along the nodes may be like the mechanism outlined for the decision tree (and random tree classifier) in FIG. 2b. However, as indicated by the dots a higher complexity of a decision tree (e.g., a higher amount of checks) may be implemented due to the above stated reasons. The feature thresholds for the number of events in the refractory period (feature A) may be in the range of 0.06 and 9.7. The feature thresholds for the kurtosis (feature B) may be in the range of 1.3 and 55. The features thresholds for the heart rate of the first cardiac vector signal (feature C) may be between 22 and 185. The feature threshold of the maximum amplitude of the first cardiac vector signal (feature J) may be between 5 and 15.

The processing unit PU may be further configured to determine an alert state. The alert state may be based on various classification results (wherein each classification result may be associated with a certain episode, i.e., time frame). For example, the alert state be based on a current classification result and the prior (i.e., last) determined classification results (e.g., a programmable number of classification results, a time window of classification results, etc.). The alert state may comprise an alert (e.g., faulty electrode detected), an advance alert (e.g., suspicious data availability, no faulty electrode yet detected), no alert (no faulty electrode detected).

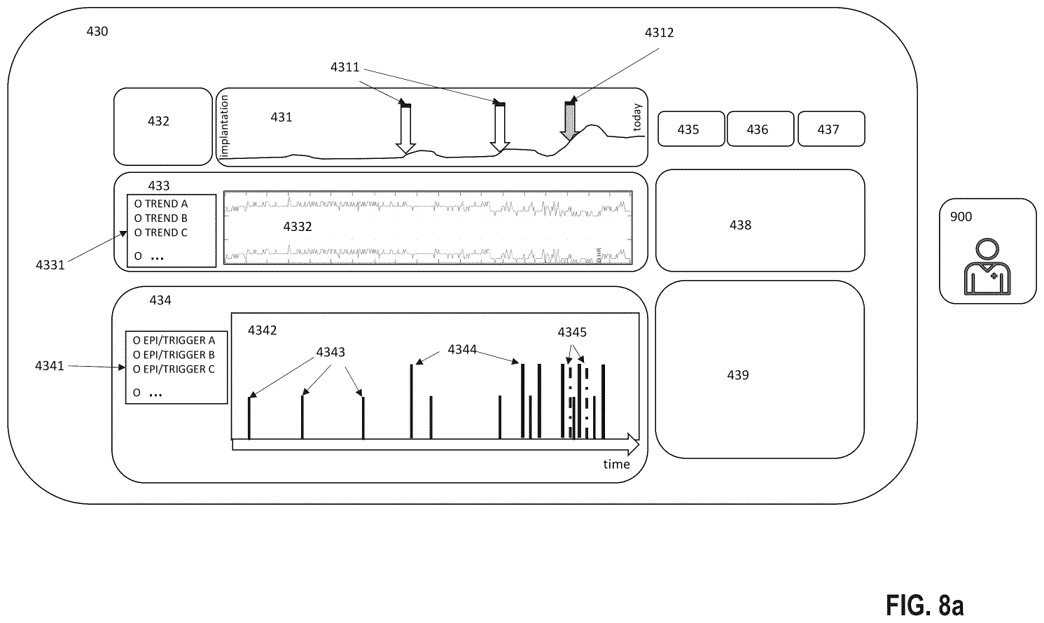

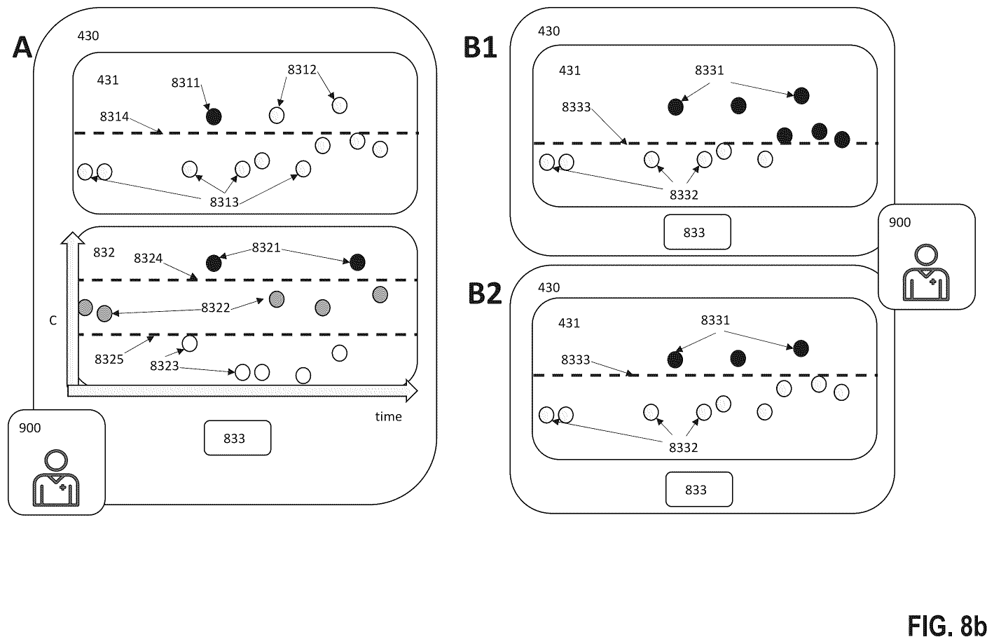

FIG. 8a shows a schematic representation of a display component 430 used in a system according to the present invention. The display component 430 may be comprised in the telemonitoring system or the programming unit 300. In this example, the display component 430 may be a user interface on a screen wherein a user (e.g., user 900) may interact with the display component 430 (e.g., by keyboard, mouse interactions, touch screen interactions, etc.). The display component 430 may comprise various modules which may indicate trends, indications, data, plots, settings, etc. The modules may also be used for input (e.g., for adapting settings, changing the indicated data, navigation, etc.). In this example, the following modules may comprise vertically aligned trend modules: a trend of total likelihood of a hardware deviation 431, a trend of supplementary information 433, a trend showing occurrences and frequency of episode types 434. The trend of total likelihood of a hardware deviation 431 may indicate the determined pₙₒᵢₛₑ (e.g., by the inventive method) over a time axis wherein the time axis may start at a date of implantation of the implantable device. The date of implantation and the current date (i.e., today) may be labeled on the axis for the user's convenience. It may be conceivable that the time axis comprises aggregated values of pₙₒᵢₛₑ for scaling purposes (e.g., an average pₙₒᵢₛₑ over a day, a week, etc.). The (pnoise) trend 431 may comprise indications of trigger events 4311, 4312. For example, it may comprise a first hardware trigger event A (e.g., an early warning 4311 regarding a suspicious electrode status) and/or a second hardware trigger event B (e.g., an alert 4312 regarding a suspicious electrode status). The trend of supplementary information 433 may comprise trends 4332 regarding the supplementary features (e.g., characteristics of daily impedance tests, pacing/stimulation threshold tests, sensing amplitude tests, heartrate and/or occurrences of unphysiological short intervals of determined heart activities, which may be labeled as TREND A, B, C . . . , etc.). The trend of supplementary information 433 may further comprise a configuration element 4331. The configuration element 4331 may be used to choose the supplementary features (e.g., from a predefined list) which are displayed in the trend 433. The trend showing occurrences and frequency of episode types 434 may comprise an episode type configuration element 4341 for choosing the episode types and trigger events (e.g., from a predefined list) which are displayed in the time axis of the trend 434. FIG. 8a further shows the time axis 4342 of the trend 434. The time axis 4342 may visualize the occurrences of the chosen episode types and trigger events (e.g., which were chosen over the episode configuration element 4341). The time axis 4342 may visualize different episode types and/or trigger events such that they visually stand apart from each other (e.g., by color, by a labeling box, by types of dashed lines, etc.). The time axis 4342 may comprise a periodic episode recording 4343, an episode recording of tachycardia 4344, a trigger event 4345.

In addition to the indicator modules the display component 430 may comprise further elements. For example, the display component 430) may comprise various indicator modules: an information module 432 about the implanted electrode system (e.g., comprising time/date of implantation, executed box changes, electrode types, etc.), an overall state 438 which may be based on a (preferably) latest alert event and all available electrode status information.

The overall state 438 may not be acknowledged by the user 500. The display component 430 may further comprise a daily module 439 which may be an indicator module showing the latest daily values/parameters (e.g., daily impedance tests, pacing/stimulation threshold tests, sensing amplitude test, frequency of unphysiologically short intervals (e.g., heart activity intervals), etc.).

The display component 430 may further comprise control elements for configuration purposes. For example, it may comprise an alert control element 435 for configuring the filter of the alert sensitivity (which will be explained in detail in FIG. 8b). The display component 430 may further comprise a pre-configuration control element 436 for storing and retrieving/requesting of pre-configurations to individually apply a configuration with “one action” to various elements (e.g., modules) of the display component 430. This may increase the usability and/or user friendliness of the display component 430. For example, if the user 900 selects a special pre-configuration from a pre-configuration menu the settings of the configuration element 4331, the episode configuration element 4341, the alert control element 435, a time control element 437. The time control element 437 comprised in the display component 430 may be used for setting the displayed range on the time axis (e.g., this may be done for the trend modules for the trend of total likelihood of a hardware configuration 431, the trend of supplementary information 433, the trend showing occurrences and frequency of episode types 433).

For example, the display component 430 may thus serve as a graphical representation of the classification results in form of a “overview-webpage”. Overall, it may serve for representing if an absolute number of trigger events (alerts, suspicious classification results, etc.) was exceeded in a certain time span. It may further serve for representing a relative increase, an absolute frequency of trigger events in a time span. It may further be configured to represent: a trend of classification results over all communicated episodes of the patient, an impedance trend (e.g., painless, daily impedance monitoring), a sense amplitude trend (e.g., from a daily measurement), a pacing threshold trend (e.g., from a daily measurement), a trend of frequency of episodes (based on their types), a VES-trend (e.g., a ventricular extra systole trend based on a daily evaluation), a LIC-trend (e.g., from a daily evaluation), a lead integrity alert trend (e.g., from a daily evaluation), follow up appointments, shock/stimulation events, the link of features may be addressed (e.g., the proportion of started charging events for shock events with regard to the number of aborted shocks). The display component 430 may enable adjusting the sensitivity of the alert. This may be implemented by deactivating (or suppressing) alerts which may be based on features which comprise a confidence value that does not exceed a parametrized confidence threshold. Further, episodes may be weighted in a specified value range of the confidence information. The display component 430 may point on its “landing page” (e.g., which may be its main overview page) to the determined “alert state” with a link option to another webpage (e.g., a link to a lead status overview webpage). In a further example, the linked webpage may be displayed on the display component 430 after the link was activated (e.g., by clicking touching the link option). In an according example, the lead status overview webpage may show the trend of the classification results of the last X days (e.g., which may be preferably 180 days).

FIG. 8b represents further examples of the display component 430 and the inventive method. In particular, FIG. 8b shows a first example A and a second example B with two exemplary versions B1 and B2. Example A may be associated with twelve classification results. In addition to displaying the classification results of pₙₒᵢₛₑ (i.e., the trend 431) a respective confidence information for each classification result may be displayed beneath the classification results. As seen in FIG. 8b, a module 832 for displaying confidence information C of the classification result of an episode evaluation may be situated underneath the trend 431.

In the first example A of FIG. 8b for each signal recording (e.g., episode) the respective confidence information C may be evaluated against two confidence thresholds in a first step. If the confidence information is below a certain lower confidence threshold 8325 the classification result may be associated with the information “weak” confidence 8323. Hence, a potential alert may be inhibited. If the confidence information is above the lower confidence threshold 8325 and below the upper confidence threshold 8324 (i.e., between the lower confidence threshold 8325 and the upper confidence threshold 8324) the respective classification result may be associated with the information “uncertain” confidence 8322. This may initiate further evaluations (e.g., evaluating if there is a frequent occurrence of multiple “uncertain” classification results in a certain time frame) wherein depending on an evaluation result an alert may be inhibited or not. Preferably, if the “uncertain” classification results are isolated episodes the alert may be inhibited. If the confidence information is at or above the upper confidence threshold 8325 (i.e., the classification results may be associated with the information “high” confidence) a (potentially) triggered alert by the classification result may not be inhibited.

In the second step of the example A the actual classification result may be checked regarding the set alert sensitivity (e.g., a check against a pₙₒᵢₛₑ threshold 8314 may be done). For example, if the pₙₒᵢₛₑ value is above a pₙₒᵢₛₑ threshold 8314 an alert may be activated. Depending on the prior confidence check (e.g., a “weak”, “uncertain” or “high” confidence) the alert may be triggered. To that regard the alert sensitivity may result from the interplay of the pₙₒᵢₛₑ threshold 8314, the lower confidence threshold 8325, and/or the upper confidence threshold 8325 with each other whereas many variations may be conceivable. The method may further comprise, that the user 900 may adjust the alert sensitivity based on the display of the confidence information 832 over a time in combination with the trend 431 of total likelihood of a hardware deviation. As explained, the hardware deviation may comprise a suspicious electrode state of the implantable device. In addition, the user 900 may adjust the alert sensitivity individually for a specific patient. In an example, the user 900 may adjust the according settings on the settings element 833. The settings element 833 may be used to configure the pₙₒᵢₛₑ threshold 8314, the lower confidence threshold 8325, the upper confidence threshold 8324 and/or the further evaluations (e.g., for the “uncertain” confidence 8322 or for further evaluations for the “weak” confidence condition 8323). Preferably, the settings element 833 may comprise a drop-down menu with pre-configured programs. The pre-configured programs may be associated with a set of parameters for an alert sensitivity. For example, the alert sensitivity may be chosen from three to seven alert sensitivity stages. The stages may correspond to a “very specific” alert sensitivity, and/or a “very sensitive” (i.e., “very reactive”) alert sensitivity.

As explained, the pₙₒᵢₛₑ values in the trend 431 may thus be associated with various trigger information for the user 900. The trend 431 may comprise pₙₒᵢₛₑ values with alert trigger information 8311 (i.e., “high” confidence and pₙₒᵢₛₑ above alert threshold) which result in an alert. It may comprise pₙₒᵢₛₑ values with a first non-trigger information 8312 (i.e., pₙₒᵢₛₑ above pₙₒᵢₛₑ threshold but “weak” or “uncertain” confidence). It may further comprise pₙₒᵢₛₑ values with a second non-trigger information 8313 (i.e., pₙₒᵢₛₑ below pₙₒᵢₛₑ threshold, hence, no alert is triggered regardless of the associated confidence information).

In the second example B of FIG. 8b the pₙₒᵢₛₑ value itself may be used to adjust the alert sensitivity. This may be accomplished by evaluating the pₙₒᵢₛₑ value against a pₙₒᵢₛₑ sensitivity threshold 8333. To that regard, if the pₙₒᵢₛₑ value exceeds the pₙₒᵢₛₑ sensitivity threshold 8333 an alert may be triggered. The versions B1 and B2 of example B show identical classification results (i.e., the trend 431 of the method but with varying pₙₒᵢₛₑ thresholds and hence varying trigger information for the classification results. The trigger information may comprise pₙₒᵢₛₑ values with a second alert trigger information 8331 (i.e., pₙₒᵢₛₑ is above the pₙₒᵢₛₑ sensitivity threshold). The trigger information may comprise pₙₒᵢₛₑ values with a second non-trigger information 8332 (i.e., pₙₒᵢₛₑ is below the pₙₒᵢₛₑ sensitivity threshold). To that regard, version B1 may be considered a setup with a relatively high alert sensitivity resulting from the comparatively low pₙₒᵢₛₑ sensitivity threshold 8333. In version B1 six of twelve classification results may be associated with the second alert trigger information 8331 (hence, only these results may initiate an alert). To that regard, version B2 may be considered a setup with a relatively low alert sensitivity resulting from the comparatively higher pₙₒᵢₛₑ sensitivity threshold 8333. In version B2 three of twelve classification results may be associated with the second alert trigger information 8331 (hence, only these classification results may initiate an alert). In the example B of FIG. 8b the settings element 833 may be used to configure the pₙₒᵢₛₑ sensitivity threshold 8333.

Conclusively, in some examples, it may be preferable that the processing unit PU is not comprised in the implantable device since a wider range of application (e.g., due to the greater computing possibilities of the method) may be possible. This may increase the detection performance, as well as enable an earlier detection of a hardware status (e.g., a hardware deviation) of the implantable device. In addition, this concept may enable that the method may be used for already implanted device (e.g., since only the data of these devices may be needed). This retrofit capability may be particularly useful for already implanted ICD-systems which may already be in connection with the telemonitoring system 400. Further, this may enable greater training capabilities on larger data sets. This may further increase the detection performance which may be verified by back-testing. To that regard a “downstream” increase of the specificity (e.g., what type of hardware deviation is present) may be mentioned in that example. For example, when the processing unit is comprised in the implantable device the method may be optimized regarding sensitivity (e.g., to optimally detect if any hardware deviation is present). This result may be used for the subsequent monitoring for a specific hardware status by the processing unit PU in the telemonitoring system 400 (e.g., when the data is available). In addition, the complex information associated with a hardware status (e.g., an electrode status) may be bundled into a unitary “overview-webpage” for an optimized usability which may improve analyzing the hardware status. To that regard, the responsible clinical personnel may be alerted early for according countermeasures. Further, the probability of inadequate therapies (including painful shocks by the implantable device) may be reduced.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Method for monitoring an implantable device, comprising:
receiving at least one cardiac vector signal, wherein the at least one cardiac vector signal is acquired by the implantable device between at least one pair of electrodes;
extracting two or more separate features from the at least one cardiac vector signal by signal processing;
deriving a hardware status (pₙₒᵢₛₑ) of the implantable device based at least in part on a classification of the extracted two or more separate features.

2. Method according to claim 1, wherein at least one of the extracted two or more separate features is based at least in part on a refractory period.

3. Method according to any one of the claim 1, wherein at least one of the extracted two or more separate features is based at least in part on a complexity of the at least one cardiac vector signal.

4. Method according to claim 1, wherein at least one of the extracted two or more separate features is based at least in part on filtering the at least one cardiac vector signal.

5. Method according to claim 1, wherein at least one of the extracted two or more separate features is based at least in part on applying a statistical analysis to the at least one cardiac vector signal.

6. Method according to claim 1, wherein at least one of the extracted two or more separate features is based at least in part on a heart rate.

7. Method according to claim 1, wherein at least one of the extracted two or more separate features is based at least in part on a plurality of received cardiac vector signals.

8. Method according to claim 1, wherein at least one of the extracted two or more separate features is based at least in part on transforming the at least one cardiac vector signal into a binary vector string based at least in part on a binary threshold value.

9. Method according to claim 1, wherein at least one of the extracted two or more separate features comprises at least one of the following features associated with the at least one cardiac vector signal:
a number of events in a refractory period, a complexity of a binary vector string, a VF-filter leakage, a kurtosis, a heart rate, an R-R interval, a maximum of a signal amplitude, a sum of signal values, a sum of signal values normalized by a maximum of the signal amplitude, a number of samples with a signal amplitude within a certain range after bandpass filtering, a first spectral moment, a mean of a frequency distribution divided by a reference peak, a maximum of an absolute autocorrelation function, a variance of a binary vector string, a number of transitions from “0” to “1” in a binary vector string, a maximum number of “0”s and/or of “1”s in a binary vector string, a phase space, a proportion of an area covered in a phase space plot, a Pearson correlation coefficient of an absolute autocorrelation function, a proportion of an area contained within a certain frequency range, a fundamental frequency.

10. Method according to claim 1, wherein the derived hardware status comprises at least one of the following:
a likelihood of a hardware deviation, a sustained technical malfunction, an electrode breakage, an intermittent technical malfunction, an external influence, an irregular external influence, an external noise, an electric hum, an interference by a medical equipment, an interference from magnetic resonance imaging.

11. Method according to claim 1, wherein the deriving is performed by an artificial intelligence system and/or machine learning system that has been trained with cardiac vector signals acquired by well-functioning and/or malfunctioning implantable devices.

12. Processing unit (PU) for monitoring an implantable device, comprising:
means for performing the method according to claim 1.

13. System for monitoring an implantable device, comprising:
an implantable device for acquiring at least one cardiac vector signal;
an external device;
wherein the implantable device and/or the external device comprises the processing unit of claim 12.

14. System according to claim 13, wherein the implantable device is configured to transmit data of at least a part of the cardiac vector signal, or data of at least a feature extracted from the cardiac vector signal to the external device,
wherein the processing unit of the external device is configured to evaluate the data of at least a part of the cardiac vector signal, or data of at least a feature extracted from the cardiac vector signal.

15. (canceled)
